(19) United States
(12) Plant Patent Application Publication
(10) Pub. No.: US 2005/0022280 P1
(43) Pub. Date: Jan. 27, 2005

(54) Osteospermum plant named 'Sunny Elizabeth'

(50) OSTEOSPERMUM ECKLONIS; OSTEOSPERMUM

(75) Inventor: Bjarne Nyholm Larsen, Odense N (DK)

Correspondence Address: FOLEY AND LARDNER;SUITE 500, 3000 K STREET NW, WASHINGTON, DC, 20007, US

(73) Assignee: SUNNY Osteospermum ApS

(21) Appl. No.: 10/873,300

(22) Filed: Jun. 23, 2004

(30) Foreign Application Priority Data
Jul. 7, 2003 (QZ) 2003/1237

(51) Int. Cl. A01H 5/00

(52) U.S. Cl. PLT/360

(57) ABSTRACT

A new and distinct cultivar of Osteospermum plant named ‘Sunny Elizabeth’, characterized by its large upright purple flower heads; flower colors: rays have stripes of RHS 155A white, and RHS 68A red-purple, with base and tip RHS 67A; dense and bushy plant form, mainly due to more upright and stronger stems, and compact plant habit; and moderately vigorous growth habit, but less need for chemical growth retardation.

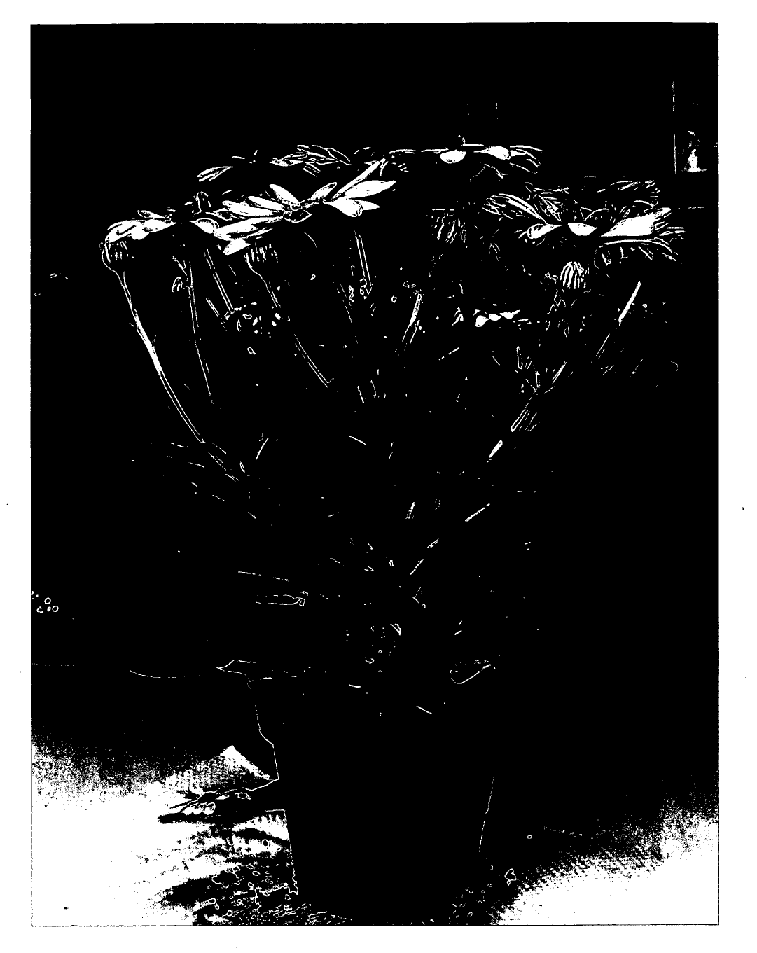

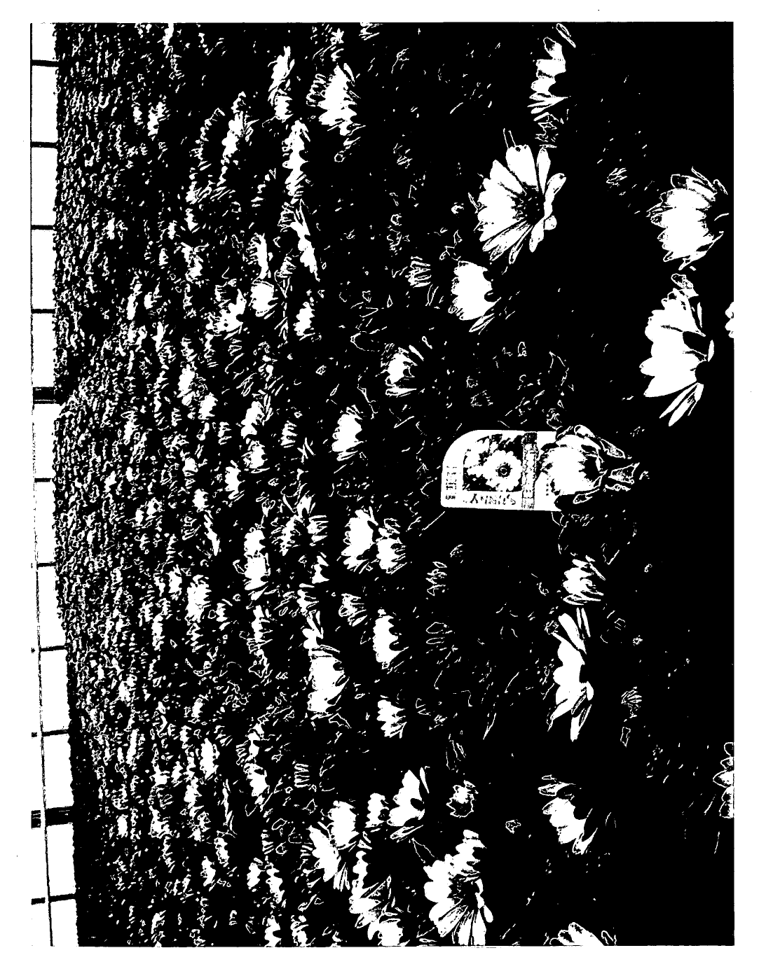

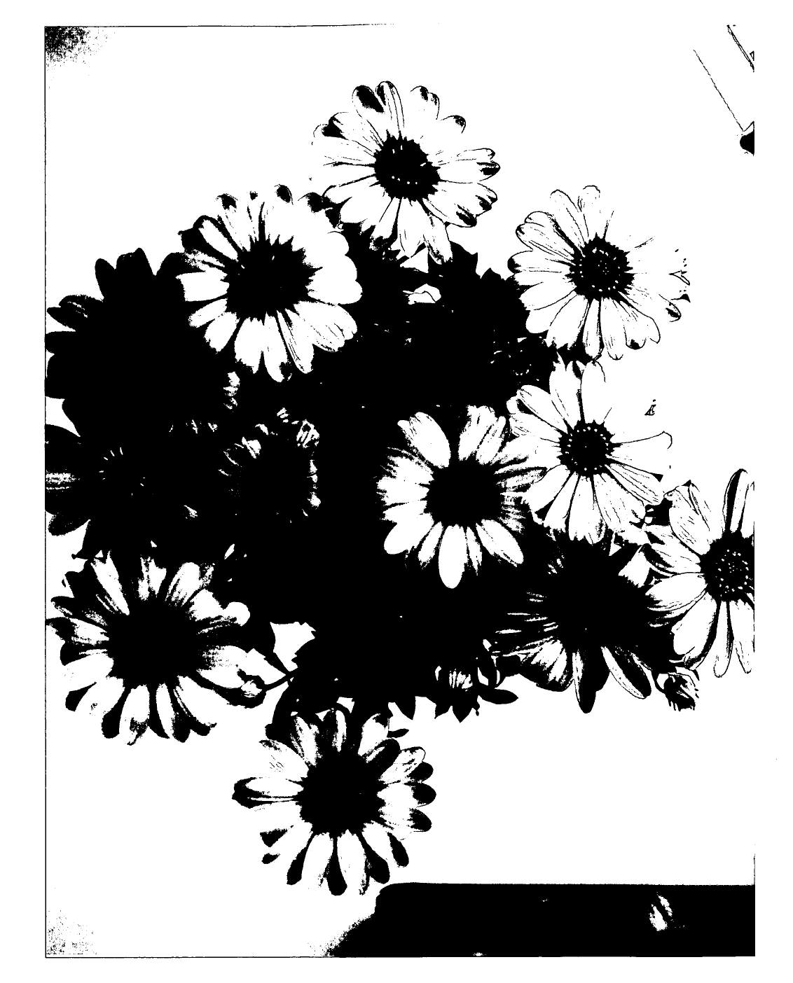

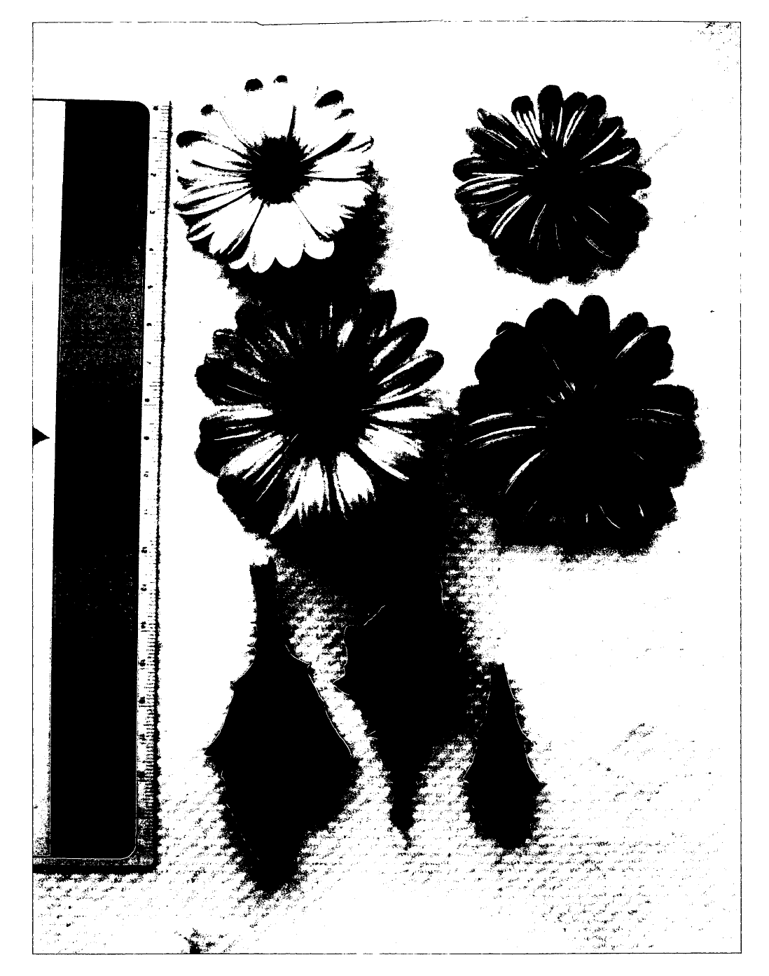

### GENUS AND SPECIES OF THE PLANT CLAIMED

Osteospermum eclonis (DC) T. Norl.

### VARIETY DENOMINATION

Sunny Elizabeth

### BACKGROUND OF THE INVENTION

The present Invention relates to a new and distinct cultivar of Osteospermum plant, botanically known as Osteospermum eclonis (DC) T. Norl., commonly known as Cape Daisy and hereinafter referred to by the variety denomination ‘Sunny Elizabeth’.

The new Osteospermum is a product of a planned breeding program conducted by the Inventor, Bjarne Larsen, in Stige, Denmark. The new Osteospermum originated from a cross made in 2000 by the Inventor between Osteospermum eclonis (DC) T. Norl. seedling designated ‘9913’ and Osteospermum eclonis (DC) T. Norl. seedling designated ‘90.017.00’. The Inventor selected the new Osteospermum cultivar from the progeny of the above crossing in 2001 on the basis of its flower color and compact, freely branching habit. Plants of the new Osteospermum are upright, compact and have a unique color combination of upper and underside of the ray petals.

Asexual reproduction of the new cultivar by terminal cuttings taken and propagated in trial production batches in Stige, Denmark, has shown that the unique features of this new Osteospermum are stable and reproduce true to type in many successive generations.

### SUMMARY OF THE INVENTION

The following traits have been repeatedly observed and are determined to be the unique characteristics of ‘Sunny Elizabeth’. These characteristics in combination distinguish ‘Sunny Elizabeth’ as a new and distinct cultivar:

1. Large upright purple flower heads, flower colors: rays have stripes of RHS 155A white, and RHS 68A red-purple, with base and tip RHS 67A;

2. Dense and bushy plant form, mainly due to more upright and stronger stems, and compact plant habit; and

3. Moderately vigorous growth habit, but less need for chemical growth retardation.

Plants of the cultivar ‘Sunny Elizabeth’ can be compared to plants of the Osteospermum eclonis (DC) T. Norl. ‘Sunny Flora’ (unpatented). In side-by-side comparisons conducted by the Inventor in Stige Denmark, plants of the cultivar ‘Sunny Elizabeth’ and the cultivar ‘Sunny Flora’ differ in the following characteristics:

1. Plants of the new Osteospermum have striking color combinations of red-purple to pink ray flowers and black disk with orange pollen.

2. Plants of the new Osteospermum have spatulate leaves while plants of the cultivar ‘Sunny Flora’ have obovate to lanceolate leaves.

3. Plants of the new Osteospermum have shorter and sturdier grey-purple peduncles while plants of the cultivar ‘Sunny Flora’ have green peduncles.

4. Plants of the new Osteospermum are shorter and more compact due to more branches than the plants of the cultivar ‘Sunny Flora’

Plants of the cultivar ‘Sunny Elizabeth’ have not been observed under all possible environmental conditions. The phenotype may vary somewhat with variations in environment such as temperature, light intensity, day length, and fertility level without, however, any variance in genotype.

### BRIEF DESCRIPTION OF THE PHOTOGRAPHS

The accompanying color photographs illustrate the overall appearance and details of flower form color and structures of the new cultivar, showing the colors as true as it is reasonably possible to obtain in color reproductions of this type. Colors in the photographs may differ slightly from the color values cited in the detailed botanical description, which more accurately describe the actual colors of the new Osteospermum. Plants of ‘Sunny Elizabeth’ were grown in 11 cm pots. The first photograph shows a side perspective view of a typical flowering plant of ‘Sunny Elizabeth’ as grown in an 11 cm pot. The second photograph shows multiple ‘Sunny Elizabeth’ plants in the greenhouse. The third photo is a close-up, top view of the composite young and older flowers of ‘Sunny Elizabeth.’ The fourth photograph shows the detail of front and back of ‘Sunny Elizabeth’ flowers as well as the front and back of the leaf.

### DETAILED BOTANICAL DESCRIPTION

In the following description, color references are made to The Royal Horticultural Society Colour Chart (RHS), 4^{th} edition. Plants were grown under greenhouse conditions. Plants used for this description were grown for about 20 weeks after cutting.
Botanical classification: Osteospermum eclonis (DC) T. Norl.
Parentage:
   Female parent.—Osteospermum eclonis (DC) T. Norl. seedling no. ‘9913’.
   Male parent.—Osteospermum eclonis (DC) T. Norl., seedling no. ‘90.017.00’.
   Propagation.—Type cutting: terminal vegetative cuttings.
   Time to initiate roots.—About 10 to 14 days at 18 to 21 C in tunnels in a greenhouse.
   Root description.—Fine, well branched.
Plant description:
   Form.—Perennial plant with upright plant habit. Osteospermum flowers in composite capitulae. Freely branching with lateral flowering branches forming at every node; dense and bushy.
   Crop time.—After rooting, about 18 weeks are required to produce finished flowering plants in 11 cm pots.
   Plant height (soil level to top of plant plane).—About 16 cm.
   Plant diameter.—21 cm.
   Vigor.—Moderately vigorous growth rate.
Foliage description:
   Leaves.—alternate, single, 4-6 lobed, brochidodromus venation. Length: 3-6 cm. Width: About 35 mm. Shape: Obovate to lanceolate. Apex: obtuse. Base: attenuate. Margin: broadly lobed. Texture: smooth, glabrous, shiny. Scattered short, stiff hairs. Color: Young foliage, upper and lower surfaces: 146B to 146C, yellow-green. Mature foliage, upper surface 147A, lower surface 147B. Venation, 148A.
Flower description:
   Flower arrangement and shape.—Tubular disk and petalled, ligulate ray flowers in composite daisy heads; with 15 parted involucre.
   Natural flowering season.—Continuous throughout the spring and summer. Season can be extended by vernalization and long day treatments.
   Flower longevity on the plant.—5 to 9 days (longevity of individual flowers is highly dependent on temperature and light conditions). Flowers persistent, petals folding and withering slowly to N187B, gray-purple.
   Inflorescence diameter.—About 6 cm.
   Flowers.—Length.—Ray petals: About 27 mm. Width: About 8 mm. Lanceolate, slightly retuse ray petal lobes. Ray petal color: Upper surfaces have stripes of 155A, white and 68A red-purple, base and tip: 67A, Disk: N89B, violet-blue. Abaxial surfaces: Disk: Tips N89A, bases 65D, red-purple.
   Ray.—Stripes of N77C, purple and 65C, red-purple (pink).
   Peduncle.—Strength: Moderately strong. Length: About 6 cm. Diameter: About 3 mm.
   Color.—Base 144A yellow-green, apical 2 cm N187A, grey.
Reproductive organs:
   Anthers.—Linear, fused, stamen color 202A, black, filament: N155B, white.
   Pollen.—N25A orange.
   Pistil and stigma.—N187A purple.
   Style.—N155B white.
   Seed.—Length about 1 mm, diameter about 0.3 mm.
Weather tolerance: Plants of the new Osteospermum have exhibited good tolerance to draught, rain and wind.

1. A new and distinct cultivar of Osteospermum plant named ‘Sunny Elizabeth’, as illustrated and described herein.
